(19) United States
(12) Patent Application Publication
(10) Pub. No.: US 2023/0314416 A1
(43) Pub. Date: Oct. 5, 2023

(54) ENHANCED BINDING OF TARGET-SPECIFIC NANOPARTICLE MARKERS

(71) Applicant: UNION BIOMETRICA, INC., HOLLISTON, MA (US)

(72) Inventors: William Peter Hansen, Drury, MA (US); Germán González Serrano, Sant Joan D´Alacant (ES); Petra B. Krauledat, Drury, MA (US)

(21) Appl. No.: 17/957,237

(22) Filed: Sep. 30, 2022

Related U.S. Application Data
(60) Provisional application No. 63/251,288, filed on Oct. 1, 2021.

(51) Int. Cl. G01N 33/53; C12Q 1/6832; G01N 33/58

(52) U.S. Cl. CPC G01N 33/5306; C12Q 1/6832; G01N 33/587

(57) ABSTRACT

Methods for enhancing the binding rate between at least two particulate binding partners are disclosed. Methods include flowing a first binding partner and a second binding partner, e.g., in a viscoelastic fluid, under conditions to chemically bind the first binding partner and the second binding partner to create a third binding partner. The flow conditions induce a particle size dependent, migration, e.g., radial, velocity differential between the first binding partner and the second binding partner and between the first binding partner and third binding partner, e.g., to increasing a collision frequency of the nanoparticles and the larger particles. Devices for enhancing the binding rate between at least two particulate binding partners are also disclosed.

### CROSS-REFERENCE TO RELATED APPLICATIONS

This application claims priority under 35 U.S.C. § 119(e) to U.S. Provisional Application Ser. No. 63/251,288, titled “ENHANCED BINDING OF TARGET-SPECIFIC NANOPARTICLE MARKERS,” filed on Oct. 1, 2021, which is hereby incorporated by reference in its entirety.

### FIELD

This disclosure concerns the enhanced binding of chemically treated nanoparticles to specific molecular targets on the surface of larger particles. The enhancement in binding is achieved by inducing size-specific differential velocities between the nanoparticles and larger particles, e.g., cells. Mechanisms of inducing such particle size dependent velocities include the use of centrifugal forces in Newtonian fluids and shear-rate gradient dependent normal forces in non-Newtonian, viscoelastic fluid flow. Such nanoparticles include sub-micron plasmonic nanoparticles employed in optically based biochemical assays. Such targets include, but are not limited to, those on an exposed surface of substantially larger particles such as supra-micron peripheral blood cells or polymeric microspheres.

### SUMMARY

In accordance with an aspect, there is provided a method for enhancing the binding rate between at least two particulate binding partners. The method may include providing a first binding partner and a second binding partner. The method may include flowing the first binding partner and a second binding partner under conditions where chemical binding between the first binding partner and a second binding partner creates a third binding partner including the first binding partner bound to a surface of the second binding partner. The flow conditions for flowing the first binding partner and a second binding partner may induce a particle size dependent, migration velocity differential between the first binding partner and the second binding partner and between the first binding partner and third binding partner.

In some embodiments, the method may include suspending the first binding partner and the second binding partner in a laminar flow viscoelastic fluid field with shear rate gradients that direct the suspended particles of the first binding partner and the second binding partner to cross laminar flow stream lines at size dependent differential transverse migration velocities.

In further embodiments, the method may include stopping fluid flow before the first binding partner, second binding partner, and third binding partner are substantially spatially separated by their transverse migration and repeating the binding reaction between the first binding partner and second binding partner.

In some embodiments, the chemical binding occurs between antigens and specific antibodies. In some embodiments, the chemical binding occurs between complimentary strands of nucleic acids. In some embodiments, the chemical binding occurs between aptamers and their associated binding partners.

In some embodiments, the first binding partners include substantially monodisperse nanoparticles. The substantially monodisperse nanoparticles may include gold or silver nanoparticles having a diameter between 5 nm to 150 nm, e.g., about 5 nm to about 20 nm, about 10 nm to about 30 nm, about 20 nm to about 40 nm, about 30 nm to about 50 nm, about 40 nm to about 60 nm, about 45 nm to about 55 nm, or about 50 nm, about 50 nm to about 100 nm, about 60 nm to about 90 nm, about 70 nm to about 80 nm, about 100 nm to about 130 nm, or about 120 nm to about 150 nm, e.g., about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, about 20 nm, about 21 nm, about 22 nm, about 23 nm, about 24 nm, about 25 nm, about 26 nm, about 27 nm, about 28 nm, about 29 nm, about 30 nm, about 31 nm, about 32 nm, about 33 nm, about 34 nm, about 35 nm, about 36 nm, about 37 nm, about 38 nm, about 39 nm, about 40 nm, about 41 nm, about 42 nm, about 43 nm, about 44 nm, about 45 nm, about 46 nm, about 47 nm, about 48 nm, about 49 nm, about 50 nm, about 51 nm, about 52 nm, about 53 nm, about 54 nm, about 55 nm, about 56 nm, about 57 nm, about 58 nm, about 59 nm, about 60 nm, about 61 nm, about 62 nm, about 63 nm, about 64 nm, about 65 nm, about 66 nm, about 67 nm, about 68 nm, about 69 nm, about 70 nm, about 71 nm, about 72 nm, about 73 nm, about 74 nm, about 75 nm, about 76 nm, about 77 nm, about 78 nm, about 79 nm, about 80 nm, about 81 nm, about 82 nm, about 83 nm, about 84 nm, about 85 nm, about 86 nm, about 87 nm, about 88 nm, about 89 nm, about 90 nm, about 91 nm, about 92 nm, about 93 nm, about 94 nm, about 95 nm, about 96 nm, about 97 nm, about 98 nm, about 99 nm, about 100 nm in diameter. In some embodiments, the nanoparticles can have a diameter of between 100 nm and 150 nm, e.g., about 101 nm, about 102 nm, about 103 nm, about 104 nm, about 105 nm, about 106 nm, about 107 nm, about 108 nm, about 109 nm, about 110 nm, about 111 nm, about 112 nm, about 113 nm, about 114 nm, about 115 nm, about 116 nm, about 117 nm, about 118 nm, about 119 nm, about 120 nm, about 121 nm, about 122 nm, about 123 nm, about 124 nm, about 125 nm, about 126 nm, about 127 nm, about 128 nm, about 129 nm, about 130 nm, about 131 nm, about 132 nm, about 133 nm, about 134 nm, about 135 nm, about 136 nm, about 137 nm, about 138 nm, about 139 nm, about 140 nm, about 141 nm, about 142 nm, about 143 nm, about 144 nm, about 145 nm, about 146 nm, about 147 nm, about 148 nm, about 149 nm, or about 150 nm.

In some embodiments, second binding partners may include cells having a diameter between 1 μm to 500 μm or polymeric microspheres having a diameter between 1 μm to 500 μm. The cells or the polymeric microspheres may have a diameter between 1 μm to 500 μm, e.g., about 1 μm to 50 μm, about 20 μm to 100 μm, about 50 μm to 150 μm, about 75 μm to 200 μm, about 100 μm to 300 μm, 150 μm to 350 μm, 200 μm to 400 μm, or about 250 μm to 500 μm, e.g., about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, or about 500 μm.

In some embodiments, the polymeric microspheres may be color coded to identify their binding specificity.

In accordance with an aspect, there is provided a method of binding nanoparticles to a surface of a larger particle. The method may include providing a suspension of nanoparticles and larger particles in a viscoelastic fluid. The method may include flowing the suspension through a lumen of a tube. A flowrate of the suspension may be chosen such that a differential radial velocity is established between the nanoparticles and the larger particles, thereby increasing a collision frequency of the nanoparticles and the larger particles.

In some embodiments, the larger particles are cells and/or polymeric microspheres, e.g., polystyrene microspheres, e.g., cells having a diameter between 1 μm to 500 μm or polymeric microspheres having a diameter between 1 μm to 500 μm. The cells or the polymeric microspheres may have a diameter between 1 μm to 500 μm, e.g., about 1 μm to 50 μm, about 20 μm to 100 μm, about 50 μm to 150 μm, about 75 μm to 200 μm, about 100 μm to 300 μm, 150 μm to 350 μm, 200 μm to 400 μm, or about 250 μm to 500 μm, e.g., about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, or about 500 μm.

In accordance with an aspect, there is provided a device for enhancing the binding rate between at least two particulate binding partners. The device may include a binding tube having a first chamber at a first end of the binding tube and a second chamber at a second end of the binding chamber. The device may include a first source of force configured to act on the first chamber and constructed and arranged, upon actuation, to create flow conditions for a sample in the binding tube that result in a first binding partner in the sample chemically binding to a second binding partner in the sample to create a third binding partner. The flow conditions from the first chamber through the binding tube may induce a particle size dependent migration velocity differential in the sample between the first binding partner and the second binding partner and between the first binding partner and the third binding partner.

In further embodiments, the device includes a second source of force configured to act on a second chamber and constructed and arranged, upon actuation, to create reverse flow conditions for a sample in the binding tube that result in a first binding partner in the sample chemically binding to a second binding partner in the sample to create a third binding partner. The flow conditions from the second chamber through the binding tube may induce a particle size dependent migration velocity differential in the sample between a first binding partner and a second binding partner and between the first binding partner and third binding partner.

In some embodiments, a diameter of the one or both of the first chamber or second chamber is greater than a diameter of the binding tube.

In some embodiments, the first chamber and the second chamber are in fluid communication with the binding tube such that the device is constructed and arranged to flow a sample from the first chamber to the second chamber. To effectuate flow of the sample from the first chamber to the second chamber, the first source of force is constructed and arranged to pressurize the sample within the first chamber to cause the sample to flow to the second chamber through the binding tube. To reverse the flow through the device, the second source of force is constructed and arranged to pressurize the sample within the second chamber to cause the sample to flow to the first chamber through the binding tube.

In accordance with an aspect, there is provided a device for enhancing the binding rate between at least two particulate binding partners. The device may include a first tube comprising a first lumen having a first proximal opening and a first distal opening. The device may include a second tube comprising a second lumen having a second proximal opening and a second distal opening. The second proximal opening may be in fluid communication with the first distal opening. The device further may include a source of a sample suspended in a viscoelastic fluid in fluid communication with the first proximal opening.

In some embodiments, a diameter of the second tube may be less than a diameter of the first tube.

In some embodiments, the second proximal opening of the second tube may be positioned centrally about an axis of flow of the viscoelastic fluid through the first tube near the first distal opening.

In some embodiments, the device may be constructed and arranged to flow the viscoelastic fluid from the first proximal opening to the first distal opening. The second tube may be positioned, e.g., at the first distal opening, and constructed and arranged to collect a concentrated stream of one or more components of the sample from the viscoelastic fluid.

In further embodiments, the device may include a receptacle disposed at the second distal opening.

### BRIEF DESCRIPTION OF THE DRAWINGS

Various aspects of one or more embodiments are discussed below with reference to the accompanying figures, which are not intended to be drawn to scale. The figures are included to provide an illustration and a further understanding of the various aspects and embodiments. The figures are incorporated in and constitute a part of this specification. But the figures are not intended as a definition of the limits of any particular embodiment. The drawings, together with the remainder of the specification, serve to explain principles and operations of the described and claimed aspects and embodiments. For purposes of clarity, not every component may be labeled in every figure. In the figures:

FIG. 1 illustrates a flow chart of a method of enhancing the binding rate between at least two particulate binding partners, according to an embodiment.

FIG. 2 illustrates a schematic of the flow of a viscoelastic fluid in a tube indicating the fluid velocity profile, shear regions, and direction of particle migration.

FIG. 3 illustrates the velocity vector components of a particle in Poiseuille flow of a viscoelastic fluid in a tube.

FIG. 4 illustrates a device for enhancing the binding rate between at least two particulate binding partner, according to an embodiment.

FIG. 5 illustrates a device for enhancing the binding rate between at least two particulate binding partner, according to another embodiment.

The features and advantages of the disclosure are apparent from the detailed specification, and thus, it is intended that the appended claims cover all systems and methods falling within the true spirit and scope of the disclosure. As used herein, the indefinite articles “a” and “an” mean “at least one” or “one or more.” Similarly, the use of a plural term does not necessarily denote a plurality unless it is unambiguous in the given context. Words such as “and” or “or” mean “and/or” unless specifically directed otherwise. In this application, the terms “comprising” and “including” may be understood to encompass itemized components or steps whether presented by themselves or together with one or more additional components or steps. Unless otherwise stated, the terms “about” and “approximately” may be understood to permit standard variation as would be understood by those of ordinary skill in the art. Where ranges are provided herein, the endpoints are included. As used in this application, the term “comprise” and variations of the term, such as “comprising” and “comprises,” are not intended to exclude other additives, components, integers or steps.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Many methodologies described herein include a step of “determining.” Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

As used herein, the term “substantially,” and grammatic equivalents, refer to the qualitative condition of exhibiting total or near-total extent or degree of a characteristic or property of interest. One of ordinary skill in the art will understand that chemical phenomena rarely, if ever, go to completion and/or proceed to completeness or achieve or avoid an absolute result.

### DETAILED DESCRIPTION

Monoclonal antibodies can be created that bind to specific antigenic structures located on the surface of biological cells. Fluorescent molecules are commonly attached to such antibodies and used as optical markers, denoting the presence of specific antigens on the cell surface. This procedure, called immunophenotyping, is often used in research and hospital flow cytometry or optical microscopy laboratories to identify cell types and function. The detection sensitivity of this method can be limited by the background fluorescence of the cell itself. Overcoming background fluorescence requires the presence of a high concentration of bound fluorescent markers per cell. Due to the high concentration needed for detection, disease-related cell surface markers can go undetected if their abundance is under a few thousand per cell. Earlier detection of disease is plausible with optically brighter markers.

It has been demonstrated that plasmonic nanoparticles, such as those made from gold or silver with diameters of approximately 50-100 nanometers (nm), can be employed under dark field microscopy conditions to replace the fluorescent molecules described above and create a light scatter based optical signal from individual plasmonic nanoparticles orders of magnitude brighter than fluorescence. With most cells, the dark field background light scatter from the cell itself is low compared to the local light scatter signal from a bound plasmonic nanoparticle, and it is possible to reliably locate as few as one bound plasmonic nanoparticle per cell. This brighter signal enables high resolution, three-dimensional, dark field optical imaging of the location of individual plasmonic nanoparticles on the cell surface. Image analysis algorithms can be used to enumerate these locations and provide a biologically useful, quantitative measure of the number of antigens per cell. This level of sensitivity enables near single antigen detection on the surface of circulating immune cells.

Due to the large size of both the nanoparticles and the cells, the use of antibody conjugated plasmonic nanoparticles to label single cells can be limited by slow particle diffusion rates and thus resulting nanoparticle-cell collision rates and binding rates are lower than with fluorescent molecules. For example, without enhancement, plasmonic nanoparticle antibody conjugates can require approximately 90 minutes to produce clinically satisfactory labeling. Alternatively, fluorochrome conjugated cell surface antigen specific antibodies typically need as little as 15 minutes to react with cells and produce satisfactory labeling.

Without wishing to be bound by any particular theory, it is believed this reaction time difference lies in the relatively low diffusion rate of plasmonic nanoparticles in liquid suspensions. The diffusion coefficient, D, for a small particle is inversely proportional to the particle hydrodynamic diameter, d. As a non-limiting example, the approximate hydrodynamic diameter of a fluorescein conjugated IgG antibody is approximately 20 nm, whereas the hydrodynamic diameter of an IgG coated 80 nm gold plasmonic nanoparticle is approximately 110 nm. The diffusion coefficient for the nanoparticle conjugate is therefore computed to be approximately 5.5 times smaller than that for the fluorescein conjugate. The time to diffuse a distance x is inversely proportional to the diffusion coefficient, D. Thus, to diffuse over the same distance requires approximately 5.5-fold less time for the fluorescein conjugated IgG antibody. This ratio of approximately 5.5 is in close agreement with the generally observed 6-fold more rapid labeling of cell surface antigens by fluorescence conjugates compared to nanoparticle conjugates. In general, cells often have hydrodynamic diameters on the order of 10 times that of an antibody conjugated nanoparticle, and thus can be considered to not diffuse at all.

Without wishing to be bound by any particular theory, it follows that other particles with surface targets, such as particles including polymeric microspheres, and such targets including surface immobilized antigens or immobilized sequences of nucleic acids, would exhibit slower binding rates for nanoparticle labeled binding partners than for fluorescence labeled binding partners. To cite a practical consequence, nanoparticle labels for polystyrene microsphere-based immunoassays and nucleic acid hybridization assays would exhibit a longer time to a result than with fluorescence labels. Therefore, there is a need to shorten the reaction time to complete a binding reaction.

Overcoming this diffusion rate discrepancy could have significant positive impact on the rate at which samples can be analyzed and especially benefit the workflow of the clinical diagnostics laboratory. The present disclosure describes how particle flow in a viscoelastic medium can be utilized to enhance the binding rate of nanoparticle conjugates to targets on the surface of substantially larger entities such as biological cells or polymeric microspheres. The present disclosure overcomes the diffusion limitation discussed above and increases the frequency of collisions between the nanoparticle conjugates, e.g., plasmonic nanoparticle conjugates, and surface targets on larger entities such as cells or polymeric microspheres. Increasing the collision frequency generally reduces the time required for labeling of the cells or polymeric microspheres.

Without wishing to be bound by any particular theory, the basis for this assertion can be justified by considering a uniform suspension of plasmonic nanoparticle conjugates and cells or polymeric microspheres in a centrifuge. In general, the laminar flow sedimentation velocity of a centrifuged particle in a liquid is proportional to the square of the particle diameter. Thus, in a mixture, the cell or polymeric microsphere speed of sedimentation can exceed the speed of the nanoparticles. In this approximation, the cells or polymeric microspheres can be viewed as passing through a suspension of nanoparticles.

Typically, one of ordinary skill would expect the nanoparticles to follow laminar flow streamlines and move aside as the cell or microsphere passes through the suspension of nanoparticles, and thus the collision rate for nanoparticles and cells or polymeric microspheres would not be increased by centrifugation. However, as described herein, the Applicant has appreciated that by including the microscopic behavior of small particles in a combined model of macroscopic and microscopic behavior, it can be shown that the nanoparticles follow streamlines only as a time-averaged behavior, and on a real time basis Brownian motion actually causes the nanoparticles to randomly depart over short distances from any given streamline, resulting in the nanoparticles colliding with passing cells or microspheres more frequently than would be the case in simple diffusion, and an increased binding rate.

Under centrifugation conditions, the settling velocity is a function of the particle diameter, the density of the particle, the density of the fluid medium, the fluid viscosity, the particle angular velocity, and the radius of the rotor for the centrifuge. As a non-limiting example, for typical peripheral blood leukocytes, e.g., about 7-15 μm in diameter, and 80 nm gold nanoparticles, the ratio of cell to nanoparticle settling speed is approximately 22:1. When antigen-specific gold nanoparticles were reacted with peripheral blood leukocytes at 300×g in a centrifuge, satisfactory labeling was achieved after 10 minutes of rotation. Compared to nanoparticle binding reactions at 1 g, i.e., without centrifugation, a close to 10-fold reduction in binding reaction time for nanoparticles was observed. This 10-minute reaction compares favorably with the 15-minute binding reaction for fluorescence conjugates as noted herein. To overcome the inherent limitations for increased binding speed, there are other physical and chemical factors that are to be addressed. There is a thin layer of fluid at the surface of the cell that can supply nanoparticles for binding to cell surface targets as cells pass through the suspended nanoparticles. This fluid layer would be quickly depleted of nanoparticles if the cell were not to move into a new region of the suspended nanoparticles, and as a result, the concentration of nanoparticles in this layer would become lower. Thus, the binding rate of nanoparticles to the cell surface would become slower if there is no replenishment of nanoparticles to this layer. Imposing a relative motion between the cell and the fluid forces the cells to travel into zones of the suspended nanoparticles where the free nanoparticle concentration therein has not been lowered by binding.

It is an object of this disclosure to provide methods and devices where the binding reaction is performed in a flow stream where a substantially uniform first mixture of freely suspended cells and freely suspended nanoparticles is introduced to a flow stream. Following the binding reaction, a substantially uniform second mixture of cells, nanoparticles, and cells with various numbers of nanoparticles bound to their surfaces can be removed from the flow stream. The second mixture of cells, nanoparticles, and cells with various numbers of nanoparticles bound to their surfaces can be achieved by use of viscoelastic suspending fluids described herein.

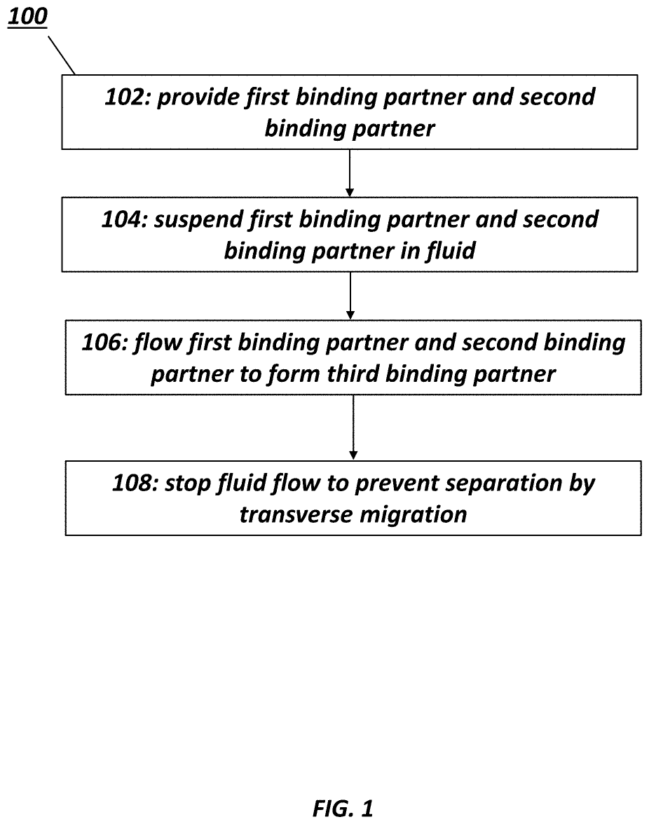

In accordance with an aspect, there is provided a method for enhancing the binding rate between at least two particulate binding partners, e.g., cells and nanoparticles. An embodiment of such a method is illustrated in FIG. 1. The method 100 includes a first binding partner and a second binding partner at step 102. The method 100 also includes flowing the first binding partner and a second binding partner under conditions where chemical binding between the first binding partner and a second binding partner creates a third particulate binding partner where the first binding partners are bound to the surface of the second binding partners at step 106. Prior to step 104, the method includes suspending the first binding partner and the second binding partner in a fluid, e.g., suspending in a laminar flow viscoelastic fluid field with shear rate gradients that direct the suspended particles of the first binding partner and the second binding partner to cross laminar flow streamlines at size dependent differential transverse migration velocities at step 104, i.e., larger particles migrate to regions of low fluid shear in the medium containing the first binding partner and a second binding partner. The flow conditions for flowing the first binding partner and a second binding partner can induce a particle size dependent migration velocity differential between the first binding partner and the second binding partner and between the first binding partner and third binding partner.

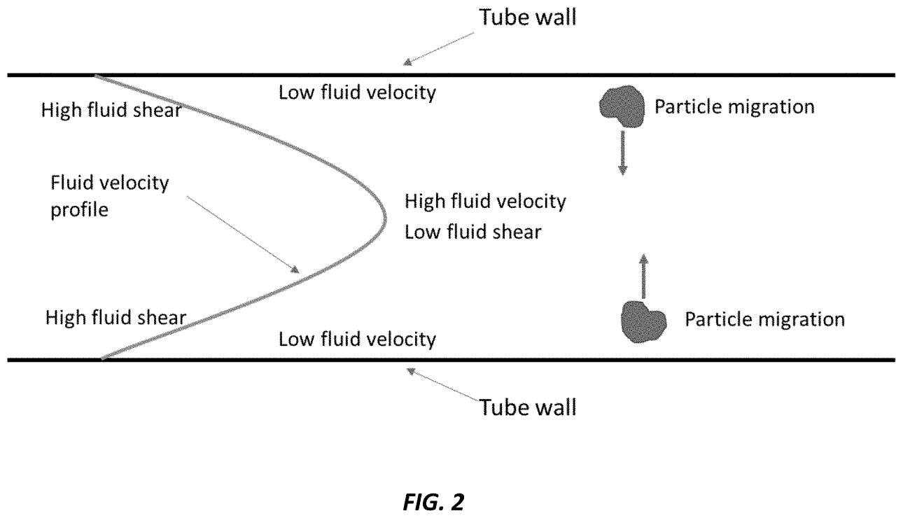

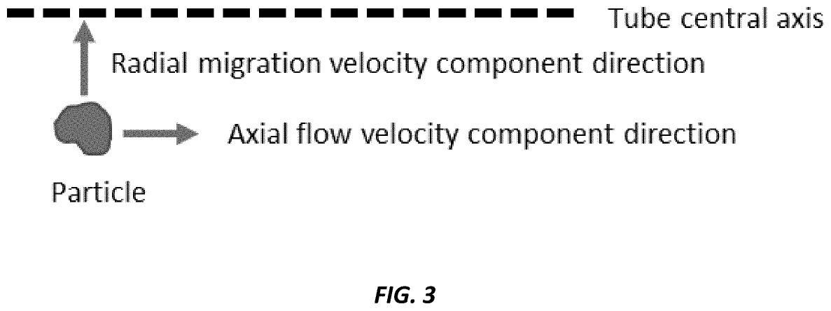

When the first binding partner and second binding partners are cells and nanoparticles, respectively, the cells and nanoparticles can be suspended in a cell-compatible, laminar flow, viscoelastic fluid. A non-limiting example of such a fluid is polyvinylpyrrolidone (PVP) in phosphate buffered saline (PBS), e.g., at a concentration of approximately 10%. When the suspension of cells and nanoparticles is directed under laminar flow conditions in a tube or lumen, as illustrated in FIG. 2, the shear rate gradient vector of the flow of the viscoelastic fluid is radial in direction, i.e., normal to the tube axis, and has a minimum at the tube central axis. The central axis of the tube is the region of lowest shear and highest fluid velocity whereas the solid inner wall of the tube is the region of highest shear and lowest fluid velocity. Thus, under these flow conditions, a flowing particle will develop an inward radial velocity component orthogonal to the central axis of the tube and an axial velocity component along the central axis of the tube as illustrated in FIGS. 2 and 3. Without wishing to be bound by any particular theory, these types of particle flow trajectories in viscoelastic fluids generally result in particles crossing streamlines and migrating toward regions of low shear rate. The streamline crossing force increases as the cube of the particle diameter. Further, in the steady state, a streamline crossing differential migration velocity is established that is proportional to the square of the particle diameter. Thus, large cells and small nanoparticles flow at different migration velocities along the direction of decreasing shear rate.

With continued reference to FIG. 1, the method 100 further includes stopping fluid flow before the first binding partner, second binding partner, and third binding partner are substantially spatially separated by their transverse migration and repeating the binding reaction between the first binding partner and second binding partner at step 108.

As disclosed herein, the methods can be used to bind numerous types of first binding partner and a second binding partner, such as antigens and specific antibodies, between complimentary strands of nucleic acids, and/or aptamers and their associated binding partners.

For example, the first binding partners can be substantially monodisperse nanoparticles, e.g., gold or silver nanoparticles having a diameter between 5 nm to 150 nm, and the second binding partners can be cells or microspheres, e.g., polymeric, e.g., polystyrene, microspheres. The microspheres can, in some embodiments, be color coded to identify their binding specificity.

Microspheres suitable for devices and methods herein can have a diameter between 1 μm to 500 μm, e.g., about 1 μm to 50 μm, about 20 μm to 100 μm, about 50 μm to 150 μm, about 75 μm to 200 μm, about 100 μm to 300 μm, 150 μm to 350 μm, 200 μm to 400 μm, or about 250 μm to 500 μm, e.g., about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, or about 500 μrn.

The monodisperse nanoparticles suitable for methods and devices disclosed herein can have a diameter of about 5 nm to 150 nm, e.g., about 5 nm to about 20 nm, about 10 nm to about 30 nm, about 20 nm to about 40 nm, about 30 nm to about 50 nm, about 40 nm to about 60 nm, about 45 nm to about 55 nm, or about 50 nm, about 50 nm to about 100 nm, about 60 nm to about 90 nm, about 70 nm to about 80 nm, about 100 nm to about 130 nm, or about 120 nm to about 150 nm, e.g., about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, about 20 nm, about 21 nm, about 22 nm, about 23 nm, about 24 nm, about 25 nm, about 26 nm, about 27 nm, about 28 nm, about 29 nm, about 30 nm, about 31 nm, about 32 nm, about 33 nm, about 34 nm, about 35 nm, about 36 nm, about 37 nm, about 38 nm, about 39 nm, about 40 nm, about 41 nm, about 42 nm, about 43 nm, about 44 nm, about 45 nm, about 46 nm, about 47 nm, about 48 nm, about 49 nm, about 50 nm, about 51 nm, about 52 nm, about 53 nm, about 54 nm, about 55 nm, about 56 nm, about 57 nm, about 58 nm, about 59 nm, about 60 nm, about 61 nm, about 62 nm, about 63 nm, about 64 nm, about 65 nm, about 66 nm, about 67 nm, about 68 nm, about 69 nm, about 70 nm, about 71 nm, about 72 nm, about 73 nm, about 74 nm, about 75 nm, about 76 nm, about 77 nm, about 78 nm, about 79 nm, about 80 nm, about 81 nm, about 82 nm, about 83 nm, about 84 nm, about 85 nm, about 86 nm, about 87 nm, about 88 nm, about 89 nm, about 90 nm, about 91 nm, about 92 nm, about 93 nm, about 94 nm, about 95 nm, about 96 nm, about 97 nm, about 98 nm, about 99 nm, about 100 nm in diameter. In some embodiments, the nanoparticles can have a diameter of between 100 nm and 150 nm, e.g., about 101 nm, about 102 nm, about 103 nm, about 104 nm, about 105 nm, about 106 nm, about 107 nm, about 108 nm, about 109 nm, about 110 nm, about 111 nm, about 112 nm, about 113 nm, about 114 nm, about 115 nm, about 116 nm, about 117 nm, about 118 nm, about 119 nm, about 120 nm, about 121 nm, about 122 nm, about 123 nm, about 124 nm, about 125 nm, about 126 nm, about 127 nm, about 128 nm, about 129 nm, about 130 nm, about 131 nm, about 132 nm, about 133 nm, about 134 nm, about 135 nm, about 136 nm, about 137 nm, about 138 nm, about 139 nm, about 140 nm, about 141 nm, about 142 nm, about 143 nm, about 144 nm, about 145 nm, about 146 nm, about 147 nm, about 148 nm, about 149 nm, or about 150 nm. An example of a nanoparticle suitable for methods disclosed herein are gold nanoparticles. While gold nanoparticles are disclosed herein, this disclosure also contemplates other plasmonic metal nanoparticles, e.g., silver nanoparticles.

The devices and methods disclosed herein are in no way limited to the pairs of binding partners disclosed herein, and any suitable and relevant binding partners can be used.

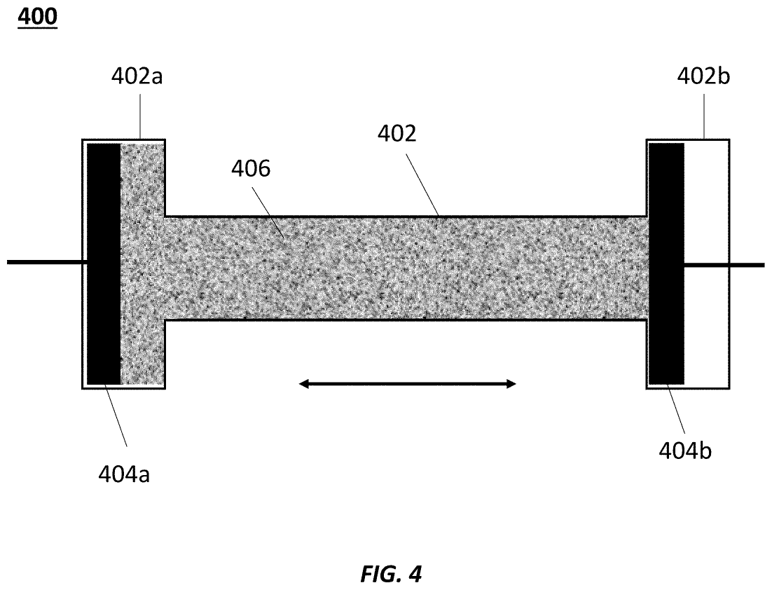

In accordance with an aspect, there is provided a device, e.g., for performing a binding reaction between a first binding partner and a second binding partner, e.g., to create a third particulate binding partner. An embodiment of such a device is illustrated in FIG. 4. The device 400 includes a binding tube 402 having a first chamber 402a positioned at a first end of the binding tube 402 and a second chamber 402b positioned at a second end of the binding tube 402. The first chamber 402a and the second chamber 402b are in fluid communication with the binding tube 402. The diameter of the first chamber 402a and second chamber 402b are greater than the diameter of the binding tube 402. Positioned within the first chamber 402a is a first source of force 404a, e.g., a first piston or another similar structural element, and positioned within the second chamber 402b is a second source of force 404b, e.g., a second piston. In operation, a sample 406 suspended in a viscoelastic fluid, e.g., a sample containing different sized components, e.g., cells and nanoparticles, is placed into the binding tube 402 through the first chamber 402a. The first source of force 404a is actuated to push the sample 406 through the first chamber 402a and binding tube 402 under pressure into the second chamber 402b, e.g., along the arrow shown in FIG. 4, to increase the collisions between the components of the sample 406. The first source of force 404a is stopped before substantial separation of the individual components of the sample 406; if separation of the individual components of the sample 406 occurs, the device 400 can be agitated, e.g., inverted or shaken, to resuspend the individual components of the sample 406. The second source of force 404b is actuated to push the sample 406 back through the second chamber 402b and binding tube 402 under pressure into the first chamber 402a, e.g., along the arrow shown in FIG. 4, with the second source of force 404b being stopped before substantial separation of the individual components of the sample 406. In further operation, the device 400 can be reagitated and the process of using the first source of force 404a and second source of force 404b to move the sample 406 within the binding tube 402 can be repeated until the binding reaction is complete, i.e., saturated. The number of repeats required is determined experimentally and may depend on the type of sample in the device being mixed. Devices such as that illustrated in FIG. 4 thus provide for an increase in collision frequency between the first and second binding partners, and therefore shorter duration binding reaction times, without the complexities of and end user skill necessitated by other techniques used for binding reactions, such as centrifugation.

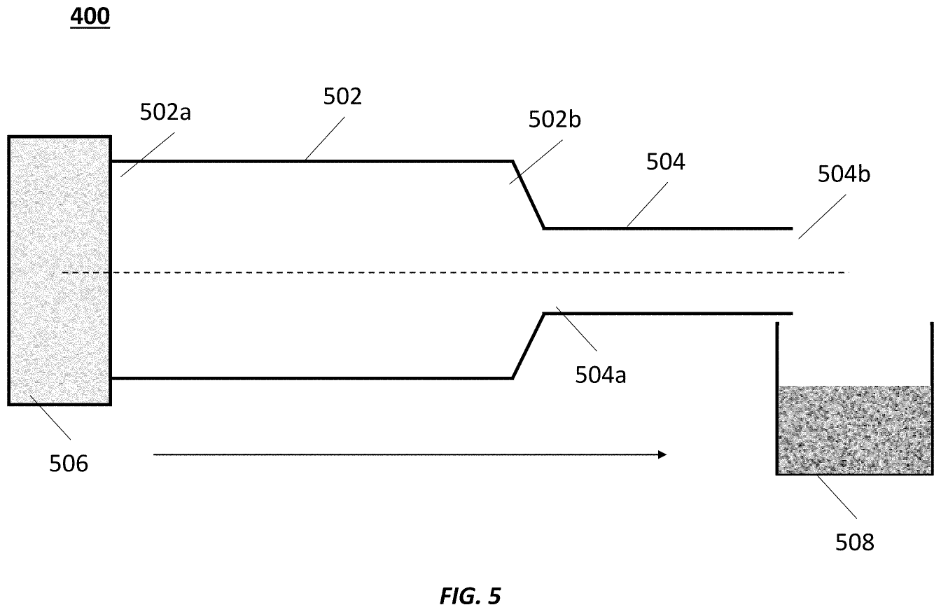

Another embodiment of a device, e.g., for performing a binding reaction between a first binding partner and a second binding partner, e.g., to create a third particulate binding partner is illustrated in FIG. 5. The device 500 includes a first tube 502 with a first lumen having a first proximal opening 502a and a first distal opening 502b. The device 500 further includes a second tube 504 having a second lumen with a second proximal opening 504a and a second distal opening 504b. As illustrated, the second proximal opening 504a is in fluid communication with the first distal opening 502b such that a fluid entering into the device 500 can pass from the first proximal opening 502a and through the second distal opening 504b along the flow direction illustrated by the arrow in FIG. 5. As illustrated in FIG. 5, the diameter of the second tube 504 is less than a diameter of the first tube 502 with the second proximal opening 504a of the second tube 404 positioned centrally about an axis of flow, e.g., the dashed line in FIG. 5, through the first tube 502 near the first distal opening 502b. In operation, a source of a sample 506 suspended in a viscoelastic fluid, e.g., a sample containing different sized components, e.g., cells and nanoparticles, is in fluid communication with the first proximal opening 504a and flows through. As the sample flows, one or more, e.g., larger, components of the sample will migrate towards the central axis of the first tube 502 and second tube 504, with the second tube 504 being constructed and arranged to collect a concentrated stream of one or more components of the sample from the viscoelastic fluid. The concentrated stream of the one or more components from the sample is collected in a receptacle 508 disposed at the second distal opening 504b.

In accordance with an aspect, there is provided a method of binding nanoparticles to a surface of a larger particle. The method includes providing a suspension of nanoparticles and larger particles in a viscoelastic fluid, e.g., PVP. The method includes flowing the suspension through a lumen of a tube at a flowrate chosen such that a differential radial velocity is established between the nanoparticles and the larger particles. The differential in radial velocity between the nanoparticles and the larger particles increases a collision frequency of the nanoparticles and the larger particles, thereby binding the nanoparticles to the larger particles. As disclosed herein, the larger particles can be cells or microspheres, e.g., polymeric, e.g., polystyrene, microspheres.

### EXAMPLES

The function and advantages of these and other embodiments can be better understood from the following examples. These examples are intended to be illustrative in nature and are not considered to be in any way limiting the scope of this disclosure.

### Example 1

In this example, the nature of Poiseuille flow of a viscoelastic fluid in a tube as a method of separating particles is explored. In this example, cells with typical sizes of 2,000 nm, i.e., 2 μm, to 20,000 nm, i.e., 20 μm, are directed through a device having a first tube and a second tube by a flowing viscoelastic fluid, such as a device illustrated in FIG. 4. As described herein, the flow of the viscoelastic fluid has high shear closest to the walls of the tube and is lowest closes to the central axis of the tube. Under these conditions, as illustrated in FIG. 5, the much larger cells move inwardly through the suspended nanoparticles toward the central axis of flow at a higher radial velocity than do the approximately 10 nm, i.e., 0.01 μm, to 100 nm, i.e., 0.1 μm, plasmonic nanoparticle conjugates. Thus, as the cells move towards the central axis of the tube, they collide with the suspended nanoparticles at a greater frequency, effectuating a faster binding reaction between relative to experiments without such flow or the complexities of centrifugation.

### Example 2

The devices and methods disclosed herein can be applied to immunoassays for soluble components present in body fluids such as serum, saliva, or urine. In particular, the devices and methods disclosed herein applies to immunoassays that are to be carried out in an aqueous environment where antibody-antigen bonds are formed that link polymeric microspheres, e.g., polystyrene microsphere, and nanoparticles, e.g., colloidal gold nanoparticles. In these types of immunoassays, polymeric microspheres having diameters of about 1-100 μm and nanoparticles having diameters of 50-100 nm exhibit low diffusion rates. As described herein for cell-nanoparticle binding, the observed lower diffusion rates slows the binding reaction between polymeric microspheres and nanoparticles. The slower binding reaction creates a long waiting time before a result of the immunoassay can be reported. The methods and devices disclosed herein suitable for cells and nanoparticles applies directly to immunoassays assays when cells are replaced by polymeric microspheres conjugated to antibodies or antigens.

### Example 3

The devices and methods disclosed herein can be applied to certain nucleic acid hybridization assays for soluble oligonucleotide targets present in an aqueous sample. In particular, the devices and methods disclosed herein can be applied to nanoparticle labeled nucleic acid probes that anneal to a complementary sequence in an oligonucleotide target and polymeric microspheres having immobilized nucleic acid probes that anneal to a second complementary sequence in the same oligonucleotide target. These two annealing reactions link nanoparticles, e.g., colloidal gold, to the surfaces of polymeric microspheres, e.g., polystyrene microspheres. In these hybridization assays, polymeric microspheres having diameters of about 1-100 μm and nanoparticles having diameters of 50-100 nm exhibit low diffusion rates. As described herein for cell-nanoparticle binding, the observed lower diffusion rates slows the binding reaction between polymeric microspheres and nanoparticles. This creates a long waiting time before a result of the hybridization assay can be reported. The methods and devices described herein for cells and nanoparticles applies directly to hybridization assays when cells are replaced by polymeric microspheres conjugated to nucleic acid probes.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure and are intended to be within the scope of the disclosure. Accordingly, the foregoing description and drawings are by way of example only.

Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed.

What is claimed is:

1. A method for enhancing the binding rate between at least two particulate binding partners, wherein:
providing a first binding partner and a second binding partner; and
flowing the first binding partner and a second binding partner under conditions where chemical binding between the first binding partner and a second binding partner creates a third binding partner comprised of the first binding partner bound to a surface of the second binding partner, wherein flow conditions for flowing the first binding partner and a second binding partner induce a particle size dependent, migration velocity differential between the first binding partner and the second binding partner and between the first binding partner and third binding partner.

2. The method of claim 1, comprising suspending the first binding partner and the second binding partner in a laminar flow viscoelastic fluid field with shear rate gradients that direct the suspended particles of the first binding partner and the second binding partner to cross laminar flow stream lines at size dependent differential transverse migration velocities.

3. The method of claim 2, further comprising stopping fluid flow before the first binding partner, second binding partner, and third binding partner are substantially spatially separated by their transverse migration and repeating the binding reaction between the first binding partner and second binding partner.

4. The method of claim 1, wherein the chemical binding occurs between antigens and specific antibodies.

5. The method of claim 1, wherein the chemical binding occurs between complimentary strands of nucleic acids.

6. The method of claim 1, wherein the chemical binding occurs between aptamers and their associated binding partners.

7. The method of claim 1, wherein the first binding partners comprise substantially monodisperse nanoparticles.

8. The method of claim 7, wherein the substantially monodisperse nanoparticles comprise gold or silver nanoparticles having a diameter between 5 nm to 150 nm.

9. The method of claim 1, wherein the second binding partners comprise cells having a diameter between 1 μm to 500 μm.

10. The method of claim 9, wherein the second binding partners comprise polymeric microspheres.

11. The method of claim 10, wherein the polymeric microspheres have a diameter between 1 μm to 500 μm.

12. The method of claim 10 or 11, wherein said polymeric microspheres are color coded to identify their binding specificity.

13. A method of binding nanoparticles to a surface of a larger particle, comprising:
providing a suspension of nanoparticles and larger particles in a viscoelastic fluid; and
flowing the suspension through a lumen of a tube, a flowrate of the suspension chosen such that a differential radial velocity is established between the nanoparticles and the larger particles, thereby increasing a collision frequency of the nanoparticles and the larger particles.

14. The method of claim 13, wherein the larger particles are cells.

15. The method of claim 13, wherein the larger particles are polymeric microspheres.

16. A device for enhancing the binding rate between at least two particulate binding partners, comprising:
a binding tube having a first chamber at a first end of the binding tube and a second chamber at a second end of the binding chamber; and
a first source of force configured to act on the first chamber and constructed and arranged, upon actuation, to create flow conditions for a sample in the binding tube that result in a first binding partner in the sample chemically binding to a second binding partner in the sample to create a third binding partner, the flow conditions inducing a particle size dependent migration velocity differential in the sample between the first binding partner and the second binding partner and between the first binding partner and the third binding partner.

17. The device of claim 16, further comprising a second source of force configured to act on a second chamber and constructed and arranged, upon actuation, to create reverse flow conditions for a sample in the binding tube that result in a first binding partner in the sample chemically binding to a second binding partner in the sample to create a third binding partner, the flow conditions inducing a particle size dependent migration velocity differential in the sample between a first binding partner and a second binding partner and between the first binding partner and third binding partner.

18. The device of claim 16, wherein a diameter of the one or both of the first chamber or second chamber is greater than a diameter of the binding tube.

19. The device of claim 16, wherein the first chamber and the second chamber are in fluid communication with the binding tube.

20. The device of claim 16, wherein the device is constructed and arranged to flow a sample from the first chamber to the second chamber.

21. The device of claim 20, wherein the first source of force is constructed and arranged to pressurize the sample within the first chamber to cause the sample to flow to the second chamber through the binding tube.

22. The device of claim 22, wherein the second source of force is constructed and arranged to pressurize the sample within the second chamber to cause the sample to flow to the first chamber through the binding tube.

23. A device for enhancing the binding rate between at least two particulate binding partners, comprising:
a first tube comprising a first lumen having a first proximal opening and a first distal opening;
a second tube comprising a second lumen having a second proximal opening and a second distal opening, the second proximal opening in fluid communication with the first distal opening; and
a source of a sample suspended in a viscoelastic fluid in fluid communication with the first proximal opening.

24. The device of claim 23, wherein a diameter of the second tube is less than a diameter of the first tube.

25. The device of claim 23, wherein the second proximal opening of the second tube is positioned centrally about an axis of flow of the viscoelastic fluid through the first tube near the first distal opening.

26. The device of claim 23, wherein the device is constructed and arranged to flow the viscoelastic fluid from the first proximal opening to the first distal opening.

27. The device of claim 26, wherein the second tube is constructed and arranged to collect a concentrated stream of one or more components of the sample from the viscoelastic fluid.

28. The device of claim 23, further comprising a receptacle disposed at the second distal opening.
